(19) United States
(12) Patent Application Publication
(10) Pub. No.: US 2008/0281165 A1
(43) Pub. Date: Nov. 13, 2008

(54) SYSTEM AND METHOD FOR ACQUIRING AND TRANSFERRING DATA TO A REMOTE SERVER

(76) Inventors: RAGHU RAI, Laguna Hills, CA (US); Michael Karian, Oceanside, CA (US)

Correspondence Address: STETINA BRUNDA GARRED & BRUCKER, 75 ENTERPRISE, SUITE 250, ALISO VIEJO, CA, 92656, US

(21) Appl. No.: 12/111,770

(22) Filed: Apr. 29, 2008

Related U.S. Application Data
(60) Provisional application No. 60/928,409, filed on May 9, 2007.

(51) Int. Cl. G06F 15/16; A61B 5/00

(52) U.S. Cl. 600/300; 709/219

(57) ABSTRACT

Systems and methods are provided for transferring data from a data acquisition device to a remote server in real time facilitated by wireless storage device operative to transmit data across a wireless network and to a portable electronic device, such as a cell phone, PDA, laptop, wireless headset or MP3 Player. Such systems and methods may be employed to transform traditional processes that require continuous analysis and monitoring to be integrated into readily available informational systems. An exemplary embodiment of the present invention is designed to streamline and facilitate the monitoring of physiological parameters so that healthcare providers may continuously monitor the physiological parameters of patients in real time.

### CROSS-REFERENCE TO RELATED APPLICATIONS

Claims priority to U.S. Provisional Application No. 60/928,409 filed on May 9, 2007 entitled: REMOTE MONITORING SYSTEM FOR PEOPLE WITH DIABETES.

### STATEMENT RE: FEDERALLY SPONSORED RESEARCH/DEVELOPMENT

Not Applicable

### BACKGROUND

The communications and computing industries are both enduring a difficult period brought on by too much success in conveying their joint potential to investors and the broader public. The relationship between these two industries, which began almost at the birth of the modern computing industry, has grown stronger and closer over the years. Just as computing devices are becoming more communications-driven, communication networks are becoming more computing-centric. As a result, content providers will likely succeed or fail based on how well they connect people in useful ways.

Currently, consumers have a variety of multimedia electronic content at their disposal. Informational content such as weather, travel tips, electronic coupons, stock exchange information, and the like are conveniently directed to consumers who are targeted to react to such content. Oftentimes, content is supplied from the provider directly to the consumer's portable electronic devices, such as personal computers, PDAs (Personal Data Assistants), cameras, MP3 players and cellular telephones, through communications services such as broadband or high-speed wireless connections. Alternative business models provide for content providers to source content at static peripheral devices or data acquisition devices. In one example of an ubiquitous commerce model, a record company sets up an eJukebox for users to connect their MP3 players for listening and downloading music directly to their portable devices.

In most portable electronic devices, memory cards, i.e., removable storage devices of one type are used in greater numbers. Attendant with most computer systems are data storage devices onto which digital data from the computer system is stored. In this manner, computer programs, textual information, graphics, and numerical data can be stored on a temporary or permanent basis for subsequent retrieval. Known as memory cards are: PCMCIA cards (PC cards) and secure digital (SD) cards and the like. SD cards have been gaining popularity in light of their compact size. Each SD card is a memory card device that incorporates flash memory. It is designed to meet demands for security, large storage capacity, and high-speed operation. Additionally, an SD card provides a security function for protecting the copyright of contents, such as music and movie data. Generally, a user may download content onto a memory card and access the content on various portable electronic devices by physically incorporating the memory card into that device.

However, the portability of such storage devices is not efficient when employed in situations that require continuous analysis and monitoring. The frequent transfer of data between an SD card to portable devices may lead to components being damaged or lost. One such situation arises in the field of telemedicine. Telemedicine is a rapidly developing application of clinical medicine where medical information is transferred via telephone, the Internet or other networks for the purpose of consulting, and sometimes remote medical procedures or examinations. In order to effectively treat certain medical conditions, it is advantageous to continuously monitor certain physiological parameters within patients.

For example, diabetic patients require ongoing assessment for determination of the level of glucose in their blood. Such test, assessment or determination is typically accomplished by an invasive procedure which, especially in the case of human diabetics, may require the drawing of blood samples, typically at least four times a day, in order to adequately monitor levels of glucose in the blood. Typically, the invasive procedure involves physically withdrawing blood from the finger tips or the ear lobes by using suitable lancing devices or withdrawing blood from veins using suitable hypodermic syringes. Once withdrawn, the blood sample is then deposited within a suitable device which determines the level of blood glucose within a certain level or accuracy and reliability.

The use of such invasive procedures to test, assess, or determine the level of blood glucose, however, is often painful, uncomfortable, frightening and overall quite undesirable. These disadvantages are exceptionally notable in the case of young children or very ill or infirm individuals who are particularly sensitive to such invasive tests and/or may be embarrassed or feel self conscious about taking blood tests. Furthermore, such invasive tests can pose the risk of infection, scaring, nerve damage, and a host of other issues associated with the repeated practices of invasively drawing blood. Indeed, such painful and traumatic practices, which must be repeatedly performed on a daily basis, at times has the effect of discouraging people afflicted with diabetes from properly testing their blood glucose levels, thereby creating a significant risk of developing serious or even life-threatening complications, not to mention shortening the person's lifespan.

Currently, there is no effective diabetes management system that effectively allows for continuous remote monitoring of diabetes in real time. Additionally, due to the sensitive nature of medical data and the importance of providing an accurate means of communication between patient and healthcare providers, it is critical that any diabetes management system is designed in a reliable and secure manner. It is equally important that any new system intended to replace an existing medical monitoring process is easy for the patient to adapt to. In this regard, systems must not only be designed to incorporate the requisite functional specifics of the process, but also possess effective data storage and transfer protocols that promote users to adopt the system as part of their daily regimen.

As a result, there is currently a need in the art for a system and method to continuously transmit data, acquired from a data acquisition device, to portable electronic devices and remote servers in a secure, reliable, and fast manner. It is further contemplated that the systems and methods should be adapted for the individuals to easily employ.

### BRIEF SUMMARY

In accordance with the present invention, there are provided multiple embodiments of systems and methods for acquiring and transferring data to a remote server. In a basic embodiment of the present invention, a system is provided for acquiring and transferring data to a remote server via a wireless storage device. The system includes a data acquisition device for acquiring data from a content source provider or from an individual. The data acquisition device subsequently transmits the data to the memory of a wireless storage device. The data transfer is facilitated through a secure wireless network, which can be configured from any wireless telecommunications platform known in the art, including any suitable cellular network, proprietary network such as Bluetooth, WiFi, or any other means known or later developed that can transmit information wirelessly. Once received by the wireless storage device, the signal can thus be transmitted to any type of portable electronic device, such as a cell phone, PDA, laptop, wireless headset, MP3 player, or the like. Subsequently, the portable electronic device may transmit the data to a remote server via the Internet for storage or for third parties to access the data.

According to an aspect of the invention, it is contemplated that a wireless data storage device capable of interfacing with any of a variety of conventional telecommunications devices, and in particular computers, notebooks and the like, will be provided that will be operative to receive, store, retrieve and transmit data across a conventional telecommunications platform. In one preferred embodiment, the wireless storage device may be a wireless micro secure digital (SD) card. A wireless micro SD card includes a package having the dimensions of a conventional memory card that includes a wireless transmitter and a memory device for storing data.

In an alternative embodiment of the present invention, the wireless storage device may take the form of a wireless USB flash drive having a key design. Such flash memory devices, which may be fashioned as a “memory stick” are readily known in the art, and may be operative to include software operative to receive, store, retrieve and transmit data associated with the data acquisition device. The wireless USB flash drive would further house a transmitter for receiving and transmitting data via a conventional telecommunications modality, such as a proprietary system like Bluetooth, or radio signals, RFID, and the like.

In an exemplary embodiment of the present invention, the data acquisition device may be a medical device being operative for measuring the physiological parameters of an individual (e.g., glucose readings, blood pressure, cholesterol, and the like). The medical device would measure and acquire physiological parameters by utilizing a biomedical sensor that is implanted on the individual. The medical device would house a computer processor having memory to store and run software for organizing and maintaining the physiological parameters. Additionally, the medical device would further house a transmitter for receiving and transmitting data via any conventional telecommunications modality to transmit the data to a wireless storage device.

It is contemplated that the portable electronic device would be operative to interface, via the Internet, with one or more servers that may be operative to upload data to the device or otherwise receive data from the device to thus enable the user or a third party to access the information. In this regard, the system is designed according to the inherent functional specifications of the data. For example, if the data acquisition device was a digital camera, pictures may be stored and categorized on online servers for the user to reference and third parties to view or comment on. Alternatively, in a medical device context, proprietary servers may permit designated physician and other healthcare providers, insurance carriers and the like to exchange information and enable the individual's medical data to be assessed for readings, trends and the like. To ensure privacy, it is contemplated that conventional security measures, such as the use of passwords and secure login identification practices are implemented.

Further in accordance with the present invention, there is a provided a method for acquiring data through a data acquisition device and transferring data to a remote server. The method initiates by utilizing a data acquisition device having an input means to acquire and store data. Subsequently, the data is transmitted from the data acquisition device to a wireless network and stored in a wireless storage device. The transmittal may occur at a predetermined periodic interval or expressly initiated by the user. The method continues by establishing a connection between a wireless storage device and a portable electronic device and initiating a data transfer such that data is transferred to the portable electronic device. The portable electronic device may be preprogrammed with proprietary data management software to assess and analyze the data. The method continues by establishing a connection between the portable electronic device and a remote secure server, via the Internet, so that data can be actively maintained and stored on the remote server in real time.

As will be appreciated, in addition to the convenience and security of transferring data to a variety of platforms by the monitoring aspects of the present invention, there is further provided a system by allows for a reliable and secure transfer of data for continuous monitoring.

The present invention is best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 depicts a basic embodiment of the present invention illustrating a data acquisition device used in conjunction with a wireless storage device to transfer data to a portable electronic device and remote server;

FIG. 2 depicts an exemplary embodiment of the present invention where the data acquisition device is a medical device configured to acquire the physiological parameters of an individual and transfer the parameters to a portable electronic device;

FIG. 2a depicts an exemplary embodiment of a biosensor configured as an arm cuff for monitoring blood pressure;

FIG. 2b depicts an alternative exemplary embodiment of a biosensor configured as a micro needle assay;

FIG. 3 depicts a wireless micro secure digital card;

FIG. 4 is a block diagram depicting a sequence of steps of acquiring data from a data acquisition device and transferring the data to a remote server.

Common reference numerals are used throughout the drawings and detailed description to indicate like elements.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

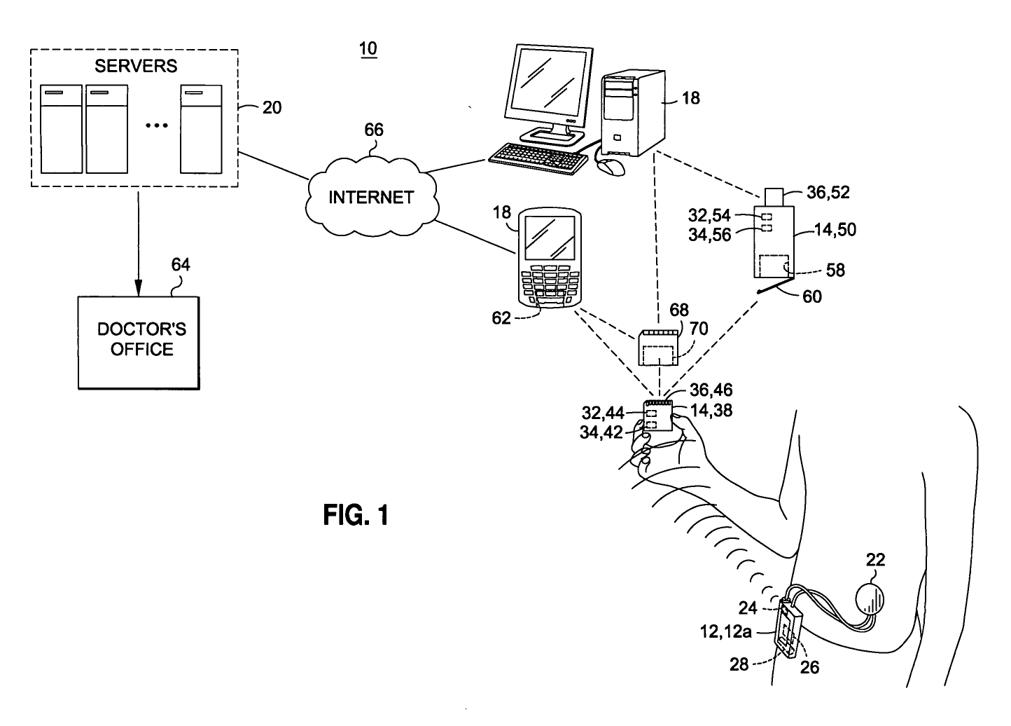

FIG. 1 depicts a system for acquiring data through a data acquisition device and transferring data to a remote server for continuous monitoring in accordance with the present invention. In a basic embodiment of the present invention, the system includes a data acquisition device 12 for acquiring data, a wireless storage device 14 to serve as an intermediate storage and transport device, a wireless network 16 for transmitting data to the wireless storage device 14, a portable electronic device 18 for accessing data on a convenient platform and further transmitting data to a remote server 20, via the internet 66.

The data acquisition device 12 is the primary source to input data in the system 10. Content providers and individual users alike may utilize the data acquisition device 12 to gather content and data for storage ultimately, in a remote server 20 or portable electronic device 18. A data acquisition device 12 may be any device capable of gathering digital data, such as a camera, MP3 player, proprietary medical devices, or the like. The data acquisition device 12 comprises an input device 22 for acquiring and entering data, a computer processor 24 having memory to store and manipulate data, and a wireless transmitter 26 to transmit data.

The input device 22 may come in a variety of formats, such as an Internet connection, or input buttons and the like. In addition, the wireless transmitter 26 may be a conventional transmitter adapted employed on complementary wireless telecommunications systems. The wireless transmitter 26 is advantageously designed so that data is transferred at predetermined periodic intervals or instantly when initiated by the user. This feature enables the device 12 to wirelessly transfer data without the user having to manually remove storage drives or establish a hard line connection with target devices. Additionally, it is contemplated that the data acquisition device 12 will come preprogrammed with proprietary software capable to manage and categorize data relative to the nature of the system 10. In this regard, if the data acquisition device 12 is configured to acquire music, the preprogrammed software may organize the data according to artist, genre, album, song, user rating or the like.

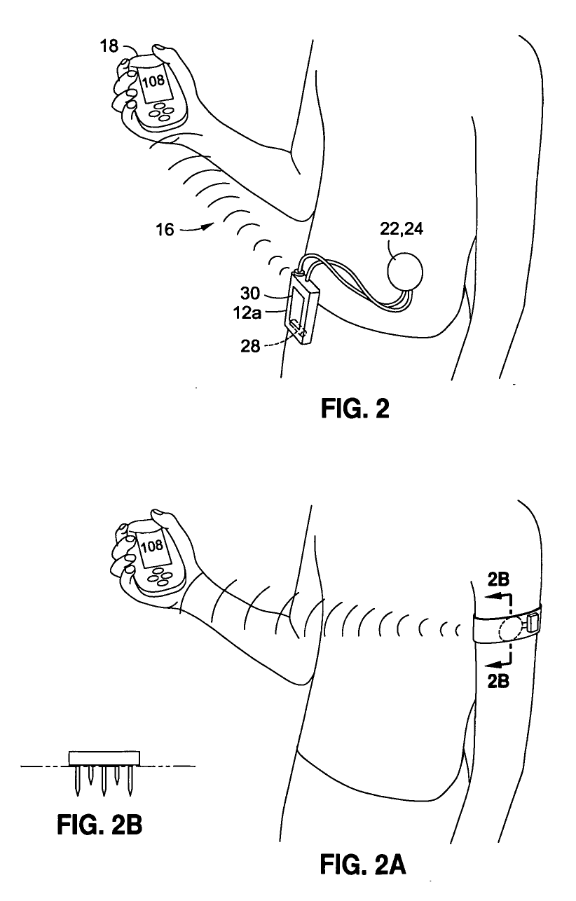

Now referring to FIGS. 1 and 2. In an exemplary embodiment of the present invention, the data acquisition device 12 is a medical device 12a being operative for measuring a physiological parameter of an individual. Physiological parameters may include glucose levels, blood pressure, heart rate and the like. A person having ordinary skill in the art will recognize that physiological parameters include any metrics taken for monitoring an individual's health. The medical device 12a further includes a biomedical sensor 24 implanted on the individual for acquiring the physiological parameter. A biosensor 24 may come in a variety of formats. In this regard, the biosensor 24 may be micro needles or nano-assays for testing glucose as illustrated in FIG. 2b, an adhesive monitor for monitoring heart rate, or an arm cuff for measuring blood pressure as illustrated in FIG. 2a. However, a person having ordinary skill in the art would understand that any appropriate medical gauge would suffice to gather the intended metric.

In addition, the medical device 22 includes a computer processor 24 having memory to store and actively maintain the physiological data. The medical device 12a may also store proprietary software, configured to run on the computer processor 24, that categorizes and analyzes the data and performs certain rudimentary medical analysis on the data, such as detect glucose levels or the like. It is contemplated that the computer software may also incorporate a calendar instructing the user to take action, such as take medication, administer a blood sample, refill a prescription, or the like. The medical device 12a further includes an electrical power source 28 (e.g., a battery component for the installation of batteries or a DC power jack/socket for use with an AC/DC adapter) for providing power to the medical device 12a. It is contemplated that the medical device 22 may further include a display screen 30 to display physiological data for the user and to provide instructions or alarms for the user to interact with. In this manner, users may monitor their physiological parameters in real time.

It is further contemplated that the medical device 12a may include an output device operative to produce a cognizable output triggered by the software program in conjunction with the physiological data (e.g., such as an alarm being triggered by high blood sugar levels). In this regard, an output device may be an internal speaker or LED lights to attract the attention of the user. A person having ordinary skill in the art will understand that the output device may be any sort indicator designed to garner the individual's attention. The medical device 12a is also equipped with a wireless transmitter 26 operative to transmit physiological data, through a secure wireless network 16, to a wireless storage device 14. The wireless transmitter 26 may be configured to transfer data automatically at periodic intervals or when initiated by the user. This feature is particularly advantageous when the system is deployed for critically ill patients, children, or any person who may have difficulty in attempting to manually transmit data. In addition, routine and consistent medical testing is critical to ascertain an accurate depiction of one's health. In this regard, it is advantageous for the system to automate the data transfer process so that potential user error is minimized.

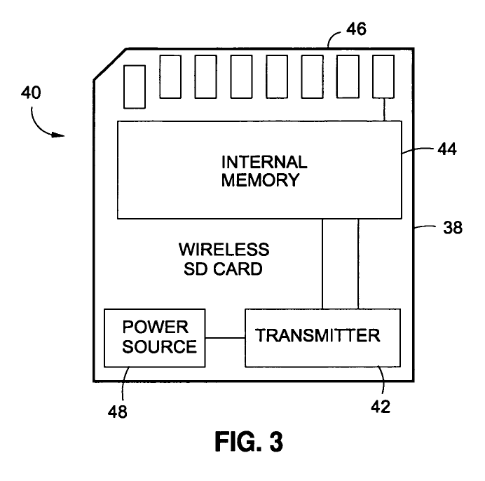

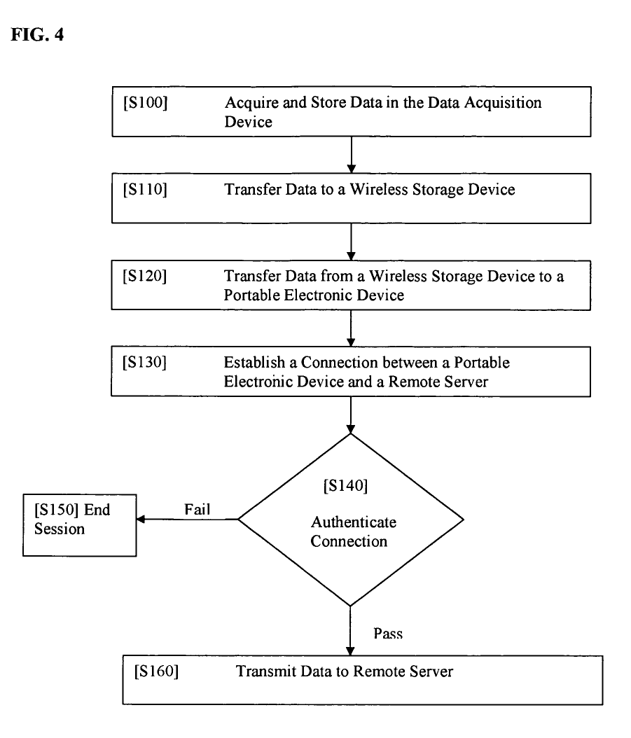

Now referring to FIG. 1, a wireless storage device 14 is configured to be operative for receiving data from the wireless secure network 16. A wireless storage device 14 includes memory 32 for storing data, a wireless transmitter 34, and a connection terminal 36 to output data. Now referring to FIGS. 1 and 3, in an exemplary embodiment of the present invention, the wireless storage device 14 is a wireless micro secure digital (SD) card 38. A wireless micro SD card 38 comprises of a package 40 that is sized in accordance to a conventional memory card such as Compact flash, SD card, PCMCIA card, XD card, and the like. In the present embodiment, the package 40 is sized in accordance with the dimensions of a conventional micro SD card with dimensions of 15 mm×11 mm×7 mm. A wireless transmitter 42 is disposed within the package 40 to receive data from the data acquisition device 12. Data is transferred from the data acquisition device 12 to the wireless micro SD card 14a through a wireless network 16 (described below). In addition, the package 40 comprises a memory device 44 being operative to store data. A memory device 44 may be any type of memory card such as Compact flash, SD card, PCMCIA card, XD card, or the like. A person having ordinary skill in the art will understand that these examples serve as exemplary embodiments and are not intended to limit the invention. In this manner, a person having ordinary skill in the art will understand that any memory card may be utilized as a memory device 44. In the present embodiment, it is contemplated that the memory card has at least 4 GB of storage space.

It is further contemplated that the memory device 44 may be partitioned so that a secure storage area is provided to store data. In the secure storage area, access may be limited to only proprietary parties monitoring the system 10. Thus rendering the data tamper proof. Providing such data security measures to the system 10 allows users to safeguard sensitive data such as medical information or proprietary software. The package 40 further includes a connection terminal 46 to transfer data to a portable electronic device 18 or an SD card adapter 68.

An SD card adapter 68 provides the wireless micro SD card 38 with versatility so that it may be operable with a variety of portable electronic devices 18. In this regard, existing portable devices 18, due to size constraints, may not be configured to employ a wireless micro SD card 38. The SD card adapter 68 may be sized in accordance to a conventional SD card so that it may be employed in a multitude of portable electronic devices 18. Additionally, the SD card adapter 68 includes a slot 70 that is configured to receive a wireless micro SD card 38. Therefore, the wireless micro SD card 38 may be incorporated into a portable electronic device 18 and initiate a data transfer. In an alternative embodiment of the present invention, it is contemplated that the wireless micro SD card 38 may be directly integrated into existing SD cards. Alternatively, it is further contemplated that a wireless micro SD card 38 may utilize wireless networks 16 to transmit data directly to a portable electronic device 18. In addition, the package 40 further includes an internal power source 48 to power the wireless transmitter 42.

Now referring to FIG. 1, in an alternative embodiment of the present invention, the wireless storage device 14 is a wireless USB flash drive 50. A wireless USB flash drive 50 is a flash memory data storage device integrated with a USB (universal serial bus) connector 52. Wireless USB flash drives 50 offer potential advantages over other storage devices. They are more compact, faster, hold more data, are more reliable for lack of moving parts, and have a more durable design. A wireless USB flash 50 drive consists of a small printed circuit board 54 typically in a plastic or metal casing and more recently in rubber casings to increase their robustness. This makes the drive sturdy enough to be carried about in a pocket, for example as a key fob, or on a lanyard. A wireless USB flash drive includes a USB connector 52 that engages with a corresponding USB port in a portable electronic device 18 for data transfer. In addition USB ports, which enable data transfers to portable electronic devices 18, appear on almost every current mainstream PC and laptops. A USB connector 52 is typically protected by a removable cap or by retracting into the body of the drive. Additionally, a wireless USB flash drives includes a wireless transmitter 56 to transmit and receive data. As illustrated in FIG. 1, a wireless micro SD card 38 may be incorporated into a wireless USB flash drive 50 to increase storage capacity or transfer data. In this regard, the wireless USB flash drive 50 includes an input socket 58 to receive the SD card 38 and a protective door 60 to secure the card in position.

To access data stored in a wireless USB flash drive 50, the drive must be connected to a USB port, either a host controller built into a computer, a USB hub, or a portable electronic device 18, such as a PDA with an USB-in port. Wireless USB flash drives 50 are active only when plugged into a USB connection and draw all necessary power from the supply provided by that connection. It is contemplated that the wireless USB flash drive 50 may be preprogrammed with proprietary data management software that automatically launches when the drive is incorporated into a portable electronic device 18. In addition, the wireless USB flash drive 50 may be configured so that the data acquisition device 12 transmits data to the drive 50 at periodic intervals. In this regard, the wireless USB drive 50 may permanently remain incorporated into a portable electronic device 18 and continuously receive data from the data acquisition device 12 as illustrated by FIG. 2. In this regard, the user may be unaware that the data is being transferred. This advantageous feature automates the transfer and storage of critical information without any user involvement. In this manner, the wireless USB drive 50 may be configured to automatically transmit data at a particular time or at the occurrence of a particular event such as when the device retrieves a predetermined amount of data or immediately after the device initiates a test. Alternatively, the wireless USB flash drive 50 may include an initiate button that permits the user to pool data instantly.

The wireless network 16 transmits data from the data acquisition device 12 to the wireless storage device 14. It is contemplated that any wireless telecommunications platform known in the art, including any suitable cellular network, proprietary network such as Bluetooth, RFID, WIFI, or any other means known or later developed that can transmit information wirelessly may be employed as the wireless network 16. In one embodiment of the present invention, the wireless network 16 is a Bluetooth network. In this regard, the wireless storage device 14 and data acquisition devices 12 are equipped with Bluetooth transmitters. Bluetooth is an industrial specification for wireless personal area networks. Bluetooth provides a way to connect and exchange information between portable electronic devices 18 such as mobile phones, laptops, computers, cameras, and the like. Bluetooth is a standard communications protocol primarily designed for low power consumption, with a short range. Additionally, Bluetooth has developed a specific range of medical frequencies that operate within the unlicensed 2.4 GHz Industrial, Scientific, Medical (ISM) range. It is contemplated that an ISM Bluetooth network would be the preferred network 16 for communications between a medical device 12a and a wireless storage device 14.

In a medical environment, an individual's physiological data is personal and highly sensitive therefore effective protection measures are critical. Additionally, most healthcare systems managing patient data must comply with HIPAA safety protocols. In this regard, Bluetooth networks employ effective security protocols to protect data. Bluetooth networks operate a feature called adaptive frequency hopping, which allows the device to identify the surrounding wavelengths and adapt its wave frequency to prevent any miscommunication or interference between the devices. In addition, Bluetooth devices employ pass code protection as a security protocol. As a result, the medical device 12a and the wireless storage device 14 are paired with a specific pass code so that they can only pair with each other. This advantageous security feature prevents unauthorized devices from improperly accessing data from the system 10. Patient data must be transmitted to parties monitoring it so that if any issues arise, the patient is immediately notified. Therefore, it is essential that patient data is transmitted to its intended target in an effective and reliable manner.

In an alternative embodiment of the present invention, the wireless network 16 is a radio frequency identification (RFID) network. In this regard, the wireless transmitter 26 of the data acquisition device 12 and the wireless transmitter 34 of the wireless storage device 14 are RFID tags. RFID is an automatic identification method, relying on storing and remotely retrieving data from RFID tags. An RFID tag generally contains two parts, an antenna and an integrated circuit for storing and processing information. RFID tags come in three general varieties: passive, active, or semi-passive. Passive tags require no internal power source and operate from a minute electrical current induced by the antenna as it receives an RF signal. However, the range of the reading is limited to 10 cm to generally two meters. An active tag contains an internal power source to initiate and engage a device for a transmission period. Active tags are considered to be more reliable and capable of transmitting signals in RF challenged environments (e.g., under water, implanted in humans or animals). A semi-passive tag contains an internal power source. However, the power is harnessed and used only to power the integrated circuit and not to broadcast a signal from the antenna.

It is contemplated that the appropriate RFID tag will be utilized based on the functional parameters of the system 10. For example, in the medical device 12a embodiment of the present invention, a biosensor 24 is implanted on an individual. In this regard, an active tag is the preferred tag so that the wireless storage device 14 can accurately and reliably detect the signal emitted from the medical device 12a. Additionally, an active tag permits a greater range of transmission, thereby providing individuals with greater mobility while retaining their connection to the system 10.

A portable electronic device 18 could range from cell phones, PDAs, laptops, wireless headsets, cameras, or any other wirelessly enabled or potentially wirelessly enabled device. Thus, the consumer can have direct, convenient, and casual access to monitor their data or transfer their data to servers for storage and accessibility to third parties. A portable electronic device 18 has memory to store data. In addition, the portable electronic device 18 is capable of establishing an Internet connection 66 to transmit data to a remote server 20. Although a wireless storage device 14 may wirelessly transmit data to a portable electronic device 18, an input connection terminal 62 is also provided so that the transfer may take place even if the network is down. In this regard, the input connection terminal 62 may be a USB in port, or a memory card connection terminal or the like.

In addition, it is contemplated that the portable electronic device 18 may be programmed to run proprietary software to analyze and manipulate data. It is contemplated that once the data is transferred to a portable electronic device 18, the user may access the proprietary software in the device 18 and view medical data, as further illustrated by FIG. 2. Additionally, the reading would represent a real-time depiction of the user's physiological state. Subsequently, the reading could be sent via traditional pathways, such as the Internet 66 to other portable electronic devices 18. The proprietary software would safely store the data in an online server under the patient's profile. In this regard, only the patient or permitted parties may access the data. It is further contemplated that if medical data is being stored or viewed on the portable electronic device 18 that HIPAA security protocols are complied with, such as a unique log-on set by the user including a user id and password.

A portable electronic device 18 is capable of transmitting data to a remote server 20 via the Internet 66. In this regard, data that is acquired may be stored in remote servers 20 for online content storage or to permit third parties to access the data. A variety of data and content storage solutions are available for storing a data. In this regard, if the data takes the form of pictures or music, online content storage servers 20 are currently available providing users with vast storage space and easy navigability to sort and categorize the data. In addition, social networking servers such as Facebook and MySpace or the like, are becoming increasingly popular for users to allow third parties to access their stored data such as photographs and music. Alternatively, it is contemplated that medical data may be stored in proprietary servers 20 that run data management software configured to monitor the physiological conditions of an individual. In this regard, the medical data is actively stored and continuously monitored over time to determine trends, patient compliance, disease management effectiveness, and the like. In addition, medical data may be transferred to interested third parties, such as a doctor's office 64, insurance companies, and the like in real time.

Further in accordance with the present invention, there is a provided a method for acquiring data through a data acquisition device 12 and transferring the data to a remote server 20. The method initiates S100 by utilizing a data acquisition device to acquire data. A data acquisition device 12 may be any device that is capable of gathering data, such as a camera, MP3 player or a medical device and the like. It is contemplated that the user may initiate the acquisition of the data, such as taking a picture with a camera, or the data acquisition device 12 may be configured to automatically gather data, such as with a medical device 12a. Subsequently, S110 the data is transmitted from the memory 24 of the data acquisition device to a wireless storage device 14 via a wireless network 16. The data may be transmitted relative to preprogrammed periodic intervals in accordance to type of wireless network deployed on the system.

The method continues S120 by connecting a connection terminal 36 of a wireless storage device to an input connection terminal 62 of a portable electronic device and initiating a data transfer so that the data is transferred to the portable electronic device 18. In this regard, the user may view, categorize, or manipulate the data. The method continues S130 by establishing a connection between the portable electronic device 18 and a remote secure server 20 via the Internet 66. Subsequently, the method continues S140 by authenticating that the connection is in accordance with established security protocols to ensure the connection is legitimate. In this manner, the server 20 is configured to employ security authentication protocols such as a user log on, password protection, or IP address verification or the like. If the authentication fails S150, the session between the portable electronic device 18 and the remote server 20 fails. However, if the authentication procedure the connection is established and the data is transmitted from the portable electronic device 18 to a remote secure server 20. Subsequently, permitted third parties are granted access to view, monitor, and analyze the data in real time. As a result, the system 10 facilitates the continuous acquisition of data and transfers the data to remote servers 20 so that third parties may monitor the data in real time.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

What is claimed is:

1. A system for acquiring and transferring data to a remote server, comprising:
a data acquisition device for acquiring and transmitting the data, the data acquisition device further including:
an input device for acquiring the data;
a computer processor having memory to store and actively maintain the data and being operative to run a data management software program which is stored in the memory;
an electrical power source for providing power to the data acquisition device;
a wireless transmitter being operative to transmit the data to a wireless network;
a wireless storage device including:
a wireless transmitter for receiving data from said wireless network;
memory for storing the data; and
a connection terminal for transferring said data to a portable electronic device;
said portable electronic device further including:
memory to store said data;
an Internet connectivity means; and
an input connection terminal for receiving the data from said wireless storage device;
a remote server that is configured for receiving the data from the portable electronic device via the Internet and having memory for storing the data.

2. The system for acquiring and transferring data to a remote server of claim 1, wherein said wireless storage device is a wireless micro secure digital card, wherein a wireless micro secure card comprises:
a package having the dimensions of a memory card, the package further including:
a wireless transmitter for receiving data from said wireless network;
a memory device incorporated in the package for storing and maintaining the data;
a connection terminal for transferring the data to said portable electronic device; and
an internal power source disposed in the package for providing power to the wireless transmitter.

3. The wireless micro secure digital card of claim 2, wherein the memory device is partitioned such that the memory device can actively store the data in a secure storage area.

4. The wireless micro secure digital card of claim 2, wherein the wireless transmitter of the wireless storage device and the wireless transmitter of the data acquisition device is a Bluetooth transmitter and the wireless network is a Bluetooth network.

5. The wireless micro secure digital card of claim 2, wherein the wireless transmitter of the wireless storage device and the wireless transmitter of the data acquisition device is a radio frequency identification tag and the wireless network is a radio frequency identification network.

6. The wireless micro secure digital card of claim 5, wherein the wireless transmitter of the wireless storage device and the wireless transmitter of the data acquisition device is a passive radio frequency identification tag.

7. The wireless secure micro digital card of claim 2, wherein the connection terminal is a standard connection terminal employed by a conventional micro SD card.

8. The system for acquiring and transferring data to a remote server of claim 1, wherein the wireless storage device is a wireless USB flash drive; wherein the wireless USB flash drive further includes:
a wireless transmitter for transmitting and receiving data;
memory for storing data and computer software; and
a conventional USB connector port for transferring data to the portable electronic device.

9. The system for acquiring and transferring data to a remote server of claim 1, wherein the a wireless USB flash drive is configured to incorporate a micro SD card.

10. The system for acquiring and transferring data to a remote server of claim 1, wherein the wireless transmitter is operative to automatically transmit the data from the data acquisition device to the portable electronic device in real time.

11. The system for acquiring and transferring data to a remote server of claim 1, wherein the remote server sends messages to the portable electronic device via the Internet.

12. The system for acquiring and transferring data to a remote server of claim 1, wherein the data acquisition device is a medical device being operative for measuring a physiological parameter of an individual, the medical device further including:
a biomedical sensor mounted on the individual for measuring and acquiring a physiological parameter;
a computer processor having memory to store and run software for converting the physiological parameter into physiological data;
an electrical power source for providing power to the medical device;
a display screen to display the physiological data; and
a wireless transmitter for transmitting the physiological data to the wireless network.

13. The system for acquiring and transferring data to a remote server of claim 12, wherein the wireless transmitter is adhesively attached to the biomedical sensor.

14. The system for acquiring and transferring data to a remote server of claim 12, wherein the medical device further includes an output device operative to produce a cognizable output triggered by the data.

15. The system for acquiring and transferring data to a remote server of claim 12, wherein the wireless transmitter is a Bluetooth transmitter and the wireless network is a Bluetooth network configured to a standard 2.4 GHz Industrial, Scientific, and Medical range.

16. A method for acquiring and transferring data to a remote server, comprising the steps:
utilizing a data acquisition device having memory to acquire data from a data source;
storing the data in the memory of the data acquisition device;
transmitting the data from the data acquisition device to a wireless storage device via a wireless network;
connecting a connection terminal of a wireless storage device to an input connection terminal of a portable electronic device and initiating a data transfer such that the data is transferred from the wireless storage device to the portable electronic device;
establishing a connection between the portable electronic device and a remote secure server via the Internet;
authenticating that the connection is in accordance with established security protocols to ensure the connection is legitimate; and
transmitting the data from the portable electronic device to a remote secure server via the Internet.

17. The method of claim 16, wherein the step of transmitting the data from the memory of the data acquisition device, the wireless storage device is a wireless micro secure digital card.

18. The method of claim 16, wherein the step of transmitting the data from the memory of the data acquisition device, the wireless storage device is a wireless USB flash drive.

19. The method of claim 16, wherein the data acquisition device is a medical device.
